(19) United States
(12) Patent Application Publication
(10) Pub. No.: US 2013/0018467 A1
(43) Pub. Date: Jan. 17, 2013

(54) Systems and Methods For Vertebral Body and Disc Height Restoration

(71) Applicant: Sean Suh, Bensalem, PA (US)

(72) Inventor: Sean Suh, Bensalem, PA (US)

(21) Appl. No.: 13/183,628

(22) Filed: Jul. 15, 2011

(51) Int. Cl. A61F 2/44

(52) U.S. Cl. 623/17.16

(57) ABSTRACT

Systems and methods are provided for assisting in the height restoration of a collapsed vertebral member, such as a vertebral body or a disc. The systems can include one or more modular spacers that are capable of being positioned within the vertebral member on their anterior and/or posterior surfaces, or on their superior and/or inferior surfaces. The methods can include forming an incision through the vertebral body and inserting one or more modular spacers through the vertebral body in a stacked configuration. The one or more modular spacers can provide a structural framework within the vertebral member and can help restore the height of the collapsed vertebral member. A bone filler material, such as a bone cement, can be injected within and around the modular spacers.

### FIELD OF THE INVENTION

The present application generally relates to intervertebral and intradiscal implants, and in particular, to modular stacking spacers.

### BACKGROUND OF THE INVENTION

The vertebrate spine is the axis of the skeleton providing structural support for the other parts of the body. Adjacent vertebrae of the spine are supported by an intervertebral disc, which serves as a mechanical cushion permitting controlled motion between vertebral segments of the axial skeleton. The intervertebral disc is a unique structure comprised of four components: the nucleus pulposus (“nucleus”), the annulus fibrosus (“annulus”) and two vertebral end plates.

Vertebral bodies can breakdown and collapse, thereby resulting in a decreased height of their bodies. The vertebral bodies can collapse overtime due to normal aging or disease. In some cases, trauma can also cause a vertebral body to collapse, such as when a load is placed on a vertebral body that exceeds its stability. Likewise, intervertebral discs can also breakdown and collapse due to normal aging, disease or trauma. Both collapsed vertebral bodies and collapsed intervertebral discs can lead to pain.

It is thus desirable to provide systems and methods that can help stabilize and restore the height of collapsed vertebral bodies and discs.

### SUMMARY OF THE INVENTION

Various systems, devices and methods are provided for vertebral body and disc height restoration. In some embodiments, a method for height restoration of a vertebral member comprises forming a first access hole through a vertebral member; inserting one or more modular spacers through the first access hole to restore height of the vertebral member; forming a second access hole through the vertebral member; and inserting one or more modular spacers through the second access hole to restore height of the vertebral member.

In some embodiments, a method for height restoration of a vertebral member comprises forming a first access hole through a vertebral member; inserting at least two modular spacers through the first access hole to restore height of the vertebral member and create a structural framework within the vertebral member; and injecting bone filler material into the vertebral member.

In some embodiments, a method for height restoration of a vertebral member comprises forming a first access hole through a vertebral member; inserting at least two modular spacers through the first access hole to restore height of the vertebral member and create a structural framework within the vertebral member, wherein the at least two modular spacers are in contact with each other within the vertebral member; and injecting bone filler material into the vertebral member.

### BRIEF DESCRIPTION OF THE DRAWINGS

FIG. 1 illustrates a perspective view of a vertebral body with a plurality of stacked spacers according to some embodiments.

FIGS. 2A-2C illustrate a method of inserting a plurality of spacers in a vertebral body according to some embodiments.

FIG. 3 illustrates a top cross-sectional view of a vertebral body with a plurality of stacked spacers and filler material according to some embodiments.

FIG. 4 illustrates a top cross-sectional view of an intervertebral disc with a plurality of stacked spacers being implanted according to some embodiments.

FIG. 5 illustrates a perspective view of an intervertebral disc with a plurality of stacked spacers according to some embodiments.

FIGS. 6A-6C illustrate a method of vertebral body height restoration according to some embodiments.

FIGS. 7A-7F illustrate an alternate method of vertebral body height restoration according to some embodiments.

### DETAILED DESCRIPTION OF THE ILLUSTRATED EMBODIMENTS

Detailed embodiments of the invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

The present application generally relates to implants such as intervertebral spacers, and in particular, to modular stacking spacers. According to some embodiments, a plurality of modular stacking spacers can be implanted into a collapsed vertebral member, such as a vertebral body and/or a collapsed intervertebral disc to restore height and stability. In some embodiments, prior to implanting the spacers, a cavity can be formed by a cavity creation element to provide a space for the spacers. The modular stacking spacers are advantageously of small size and can be delivered minimally invasively through a small incision. In addition, the stacking spacers can advantageously serve as a customizable framework within the vertebral body and/or disc to accommodate bone filler material.

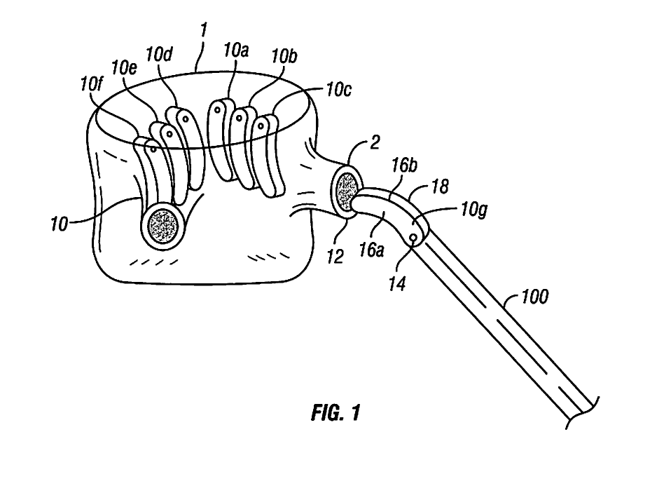

FIG. 1 illustrates a perspective view of a vertebral body with a plurality of stacked spacers according to some embodiments. A plurality of spacers 10, each individually denoted as 10a-10f, are positioned within the vertebral body 1 to assist in restoring the height of the vertebral body. Another spacer 10g is shown being inserted through the pedicle 2 of the vertebral body 1 via instrument 100. One skilled in the art will appreciate that the spacer and instrument can be inserted into a desired position within the body using a working or guiding cannula (not shown). The additional spacer 10g can be positioned within the vertebral body to further assist in height restoration.

The individual spacers 10 are advantageously modular spacers of small size that are capable of being “stacked” or otherwise positioned next to each other within the vertebral body 1. While the spacers are illustrated as being inserted through the pedicle 2, in other embodiments, the spacers can be inserted extra-pedicular. In some embodiments, a portion of one modular spacer can contact another within the vertebral body. Each of the spacers include an anterior portion 12, a posterior portion 14, top or superior portion 16a, bottom or inferior portion 16b, and curved sidewalls 18. While the spacers are illustrated as having these particular features, other features are also possible. For example, spacers having non-curved sidewalls can be provided. In some embodiments, the spacers can be configured such that a height of an anterior portion is greater than a height of a posterior portion, or vice versa.

The individual spacers 10 can be advantageously sized such that multiple spacers can be placed within a vertebral body 1. In some embodiments, the spacers 10 can have a width of between about 1 and 11 mm, a length of between about 5 and 90 mm, and a height of between about 1 mm and 11 mm. In some embodiments, the spacers 10 can have a round cross-section, while in other embodiments, the spacers 10 can have a rectangular cross-section or any other non-round cross-section. Due to the small dimensions, the modular spacers can be inserted minimally invasively through one or more small incisions. In some embodiments, the modular spacers can also be used in open and mini-open procedures as well.

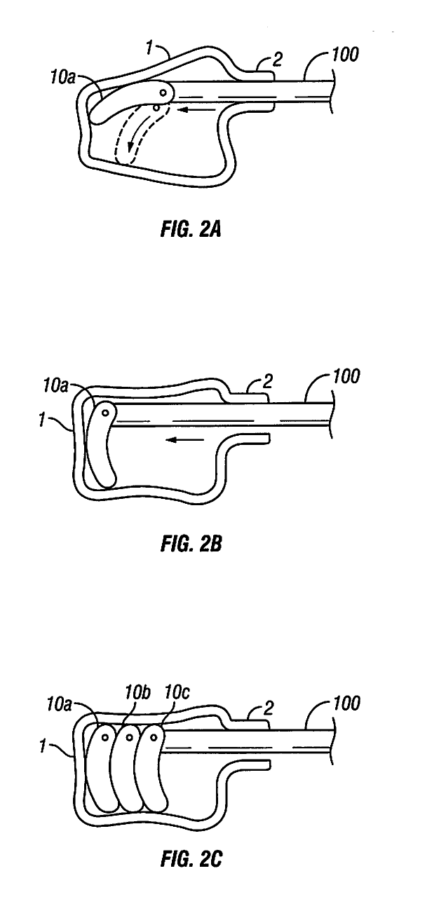

As shown in FIG. 1, the individual spacers 10 can be stacked or positioned such that multiple spacers are adjacent one another. For example, spacers 10a-10c are stacked adjacent one another, while spacers 10d-10f are stacked adjacent one another. Within the vertebral body 1, the spacers 10 are in a “vertical” stacked position (shown in FIGS. 2B and 2C), wherein the anterior portion 12 and/or posterior portion 14 of the spacers contact the walls of the vertebral body 1. In other embodiments, the spacers 10 can be in a “horizontal” stacked position (not shown) within the vertebral body, wherein the superior and/or inferior surfaces of the spacers contact the walls of the vertebral body. In addition, the spacers 10 can be stacked diagonally depending on the orientation of the cavity created. In some embodiments, multiple spacers can be stacked on top of each other in a horizontal stacked position to assist in restoring the height of the vertebral body.

The number of modular spacers 10 within a vertebral body 1 can vary. While the illustration in FIG. 1 shows at least six spacers placed within the vertebral body 1, in other embodiments, the number of spacers can be one, two, three, four, five or greater than six spacers. In addition, while the spacers in FIG. 1 are illustrated as being of similar size and shape, one skilled in the art will appreciate modular spacers of multiple sizes and/or shapes can be used to help restore the height of a vertebral body.

In some embodiments, once within the vertebral body, the plurality of spacers in the vertebral body 1 can be attached or interlocked to each other. In some embodiments, one or more spacers can have interdigitating or complementary features or textures on their surfaces to assist in the attachment or interlocking. In other embodiments, the plurality of spacers in the vertebral body 1 are not attached to each other, but are still capable of providing a strong framework to assist in vertebral body height restoration.

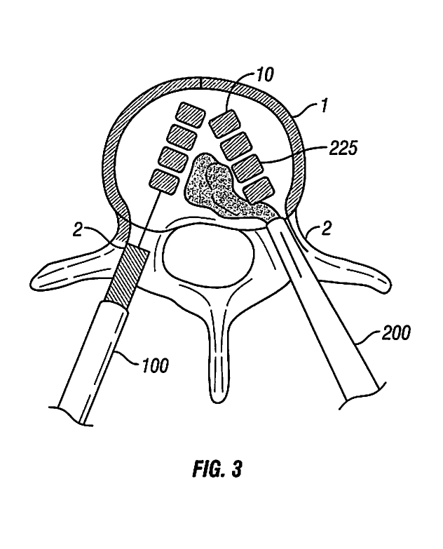

In addition to helping to restore the height of a collapsed vertebral body 1, the stacked modular spacers 10 can also advantageously provide a structural framework for which filler material can be inserted (as shown in FIG. 3). Using multiple spacers 10, the framework is advantageously customizable for each individual vertebral body. While it is possible to use a single spacer 10 in some embodiments to restore disc height, the use of modular spacers 10 reduces the need to carefully size single spacers prior to insertion within a vertebral body, as the modular spacers 10 can simply be introduced one at a time until a desired height restoration and/or structural framework is achieved.

FIGS. 2A-2C illustrate a method of inserting a plurality of spacers in a vertebral body according to some embodiments. The spacers 10 can be inserted one at a time using an instrument 100 to both deliver and release a spacer in a desired location within a vertebral body.

FIG. 2A illustrates the step of introducing a first modular spacer 10a into a collapsed vertebral body 1. The modular spacer 10a is attached at its posterior portion 14 to an instrument 100. The instrument 100 can be used to deliver the modular spacer 10a into a vertebral body 1, such as through a pedicle 2 or extrapedicular. The instrument 100 can be configured to secure and lock the spacer 10a in a first orientation during delivery. Once the spacer 10a is placed within the vertebral body, the instrument 100 can be configured to allow rotation or pivoting of the spacer 10a such that it can be oriented in a desired position within the vertebral body. As shown in FIG. 2A, the modular spacer 10a can be oriented into a desired position by using force from the instrument 100 to push the spacer 10a against the walls of the vertebral body 1. In other embodiments, the instrument 100 can include mechanical features (e.g., gears, threads, wires, etc.) that will force rotation of the modular spacer 10a into a desired orientation.

FIG. 2B illustrates the first modular spacer 10a being delivered in a desired orientation within the vertebral body by using instrument 100. In some embodiments, the spacer 10a can assume a desired position merely by using the force of the instrument 100 to push the spacer 10a against the walls. As shown in FIG. 2B, instrument 100 can be used to place spacer 10a in a vertical position, wherein its anterior portion and/or posterior portion rest on sidewalls of the vertebral body. Once the first spacer 10a is in a desired position, additional spacers can be introduced into the space of the vertebral body if desired. As shown in FIG. 2B, even a single spacer 10a can help to restore disc height.

FIG. 2C illustrates the instrument 100 delivering an additional modular spacer 10c to a desired positioned within the vertebral body. As shown in FIG. 2C, the modular spacer 10c can be stacked against or positioned adjacent a second modular spacer 10b, which is stacked against or positioned adjacent a third modular spacer 10c. In some embodiments, modular spacers 10a-10c can be fixedly attached or interlocked to one another, while in other embodiments, the modular spacers 10a-10c are not attached to one another. The three modular spacers 10a-10c help to restore disc height of the vertebral body 1. In addition, the three modular spacers 10a-10c provide a structural framework for which filler material, such as bone cement, can be introduced into the vertebral body. The modular spacers can be in any orientation with respect to each other. The modular spacers advantageously provide structural support in addition to the filler material in order to restore the height of the collapsed vertebral body 1.

FIG. 3 illustrates a top cross-sectional view of a vertebral body with a plurality of stacked spacers and filler material according to some embodiments. As shown in FIG. 3, a plurality of modular spacers 10 are introduced into a vertebral body 1 through pedicle 2 via instrument 100. The spacers 10 provide a structural framework for the filler material 225. Once the spacers 10 are in a desired position, the filler material 225 can be introduced (e.g., through a second pedicle 2) via injection instrument 200. The filler material 225 can harden within the vertebral body 1, thereby helping to restore stability and height.

Various types of filler material can be used, including natural and synthetic bone filler material. In some embodiments, the filler material includes one or more bone growth inducing agents and/or curing agents.

In addition to assisting in the restoration of height for vertebral bodies, the modular spacers described herein can also be used to assist in a corpectomy, in which a vertebral body is completely removed. The modular spacers can act as substitutes for one or more removed vertebral bodies. Furthermore, the modular spacers can also be used for the restoration of height for collapsed intervertebral discs.

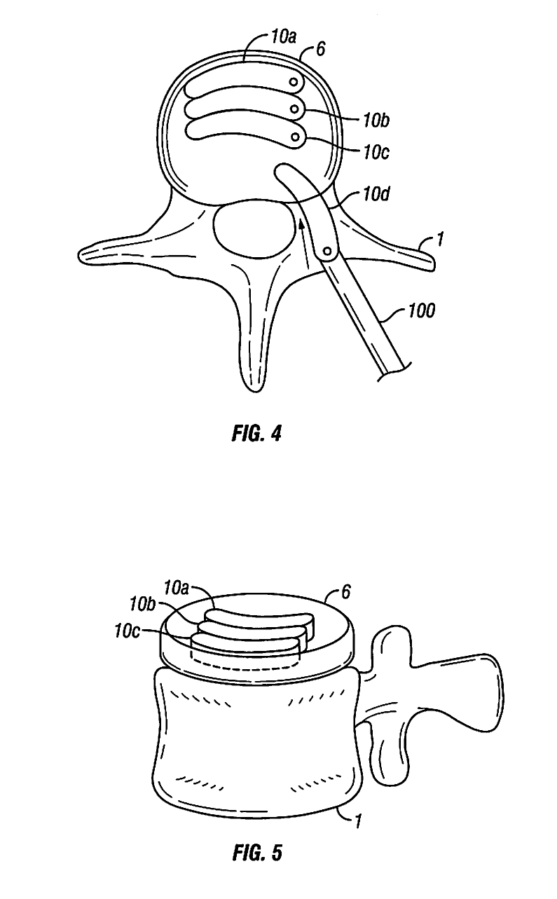

FIG. 4 illustrates a top cross-sectional view of an intervertebral disc with a plurality of stacked spacers being implanted according to some embodiments. As shown in the illustrated embodiment, at least three modular spacers 10a, 10b, and 10c have been positioned within the intervertebral disc space, while a fourth modular spacer 10d is being introduced via instrument 100. The one or more spacers can be introduced into a disc space via any number of approaches, including anteriorly, translaminarly, posteriorly, through end plates, etc. In addition, while in the illustrated embodiment, the spacers used are introduced through the disc space, in other embodiments, the spacers can be introduced through a vertebral body and into a disc space (e.g., diagonally) if desirable. Like the spacers in the vertebral body of FIG. 1, the modular spacers 10 can be stacked or positioned next to each other within the intervertebral disc 6. However, in contrast to the spacers in the vertebral body, the spacers in the disc space are illustrated as being flat on at least one of their superior and/or inferior surfaces.

While the illustrated embodiment in FIG. 4 illustrates at least three modular spacers implanted within the disc space, in other embodiments, one, two, four, five, six or more spacers can be used to assist in height restoration of the collapsed disc. In some embodiments, the modular spacers that are implanted within the disc space are of similar size and shape as those that are implanted within the vertebral bodies, while in other embodiments, the spacers are of different size and/or shape.

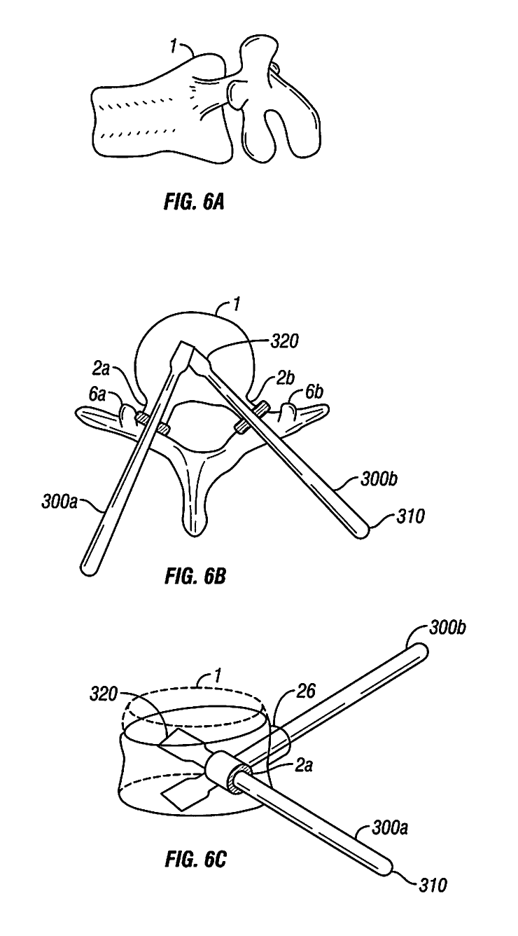

FIG. 5 illustrates a perspective view of an intervertebral disc with a plurality of stacked spacers according to some embodiments. In the illustrated embodiment, three spacers are used to restore the height of a collapsed disc. The three spacers are all of similar size and are positioned within the disc space on a superior and/or inferior surface. The three spacers provide a structural framework within the disc to provide height restoration.

### Methods of Vertebroplasty or Kyphoplasty

In some embodiments, the modular spacers can be used to assist in a vertebroplasty procedure. Methods of vertebroplasty using the modular spacers can comprise:

a. forming an incision in a body of a patient;

b. forming a hole through a portion of the vertebral body (e.g., a pedicle) to reach the inside of the vertebral body;

c. inserting an instrument through the hole to remove portions of the vertebral body and/or create a cavity or void in the vertebral body;

d. inserting a first spacer through the hole to serve as part of a structural framework within the vertebral body;

e. inserting one or more additional spacers through the hole to be positioned or stacked against the first spacer, thereby creating the structural framework within the vertebral body; and

f. injecting a bone filler material through the hole to supply bone filler around and about the structural framework created by the spacers within the vertebral body.

The modular spacers can be any of the spacers described above. In addition, the spacers can be positioned within the vertebral body in any orientation, such as on their anterior and/or posterior surfaces, or on their superior and/or inferior surfaces. One skilled in the art will appreciate that the steps performed above can be performed in different orders. For example, bone filler material can be injected prior to or during the insertion of one or more spacers through the hole, in addition or instead of after forming the structural framework.

### Additional Methods for Height Restoration of Vertebral Members

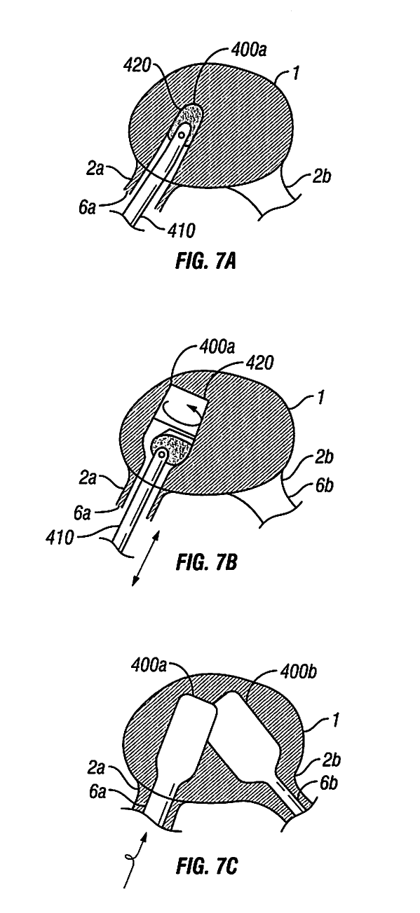

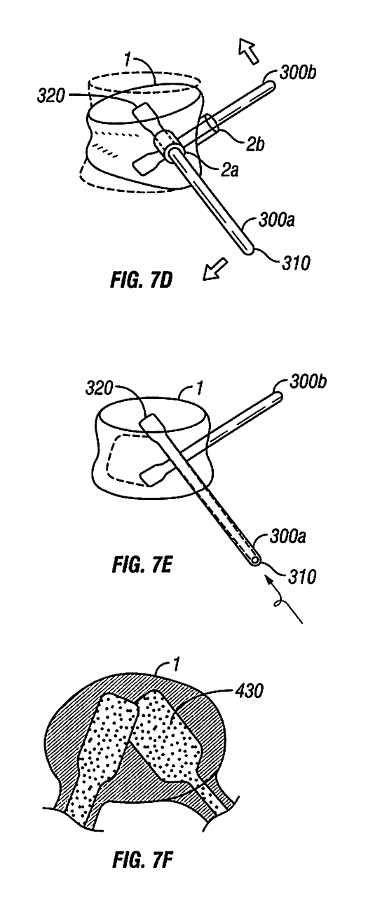

In addition to the methods described above for restoring the height of collapsed vertebral members such as vertebral bodies and discs using modular spacers, other methods are provided for which the use of modular spacers is optional. These methods are described below with respect to FIGS. 6A-7F.

FIGS. 6A-6C illustrate a method of vertebral body height restoration according to some embodiments. In the illustrated method, height restoration instruments 300a and 300b are used to restore the height of a collapsed vertebral body 1. The height restoration instruments 300a and 300b can be of various shapes and sizes. In some embodiments, the height restoration instruments 300a and 300b can be straight, straight but curvable, or curved. In some embodiments, the height restoration instruments are curvable via a shape-memory material. In some embodiments, the height restoration instruments 300a and 300b can also be cannulated to assist in the delivery of cement.

FIG. 6A illustrates a collapsed vertebral body 1. The collapse of the vertebral body can be due to aging, disease or trauma.

FIG. 6B illustrates the collapsed vertebral body 1 in FIG. 6A with height restoration instruments 300a and 300b inserted therein. Holes can be made through a portion of the vertebral body 1 to access the internal region of the vertebral body 1. In the illustrated embodiment, a pair of holes 6a and 6b are made through the pedicles 2a and 2b, although holes can be formed through other areas of the vertebral body as well, including extra-pedicularly. Once the access holes 6a and 6b have been formed, the pair of height restoration instruments 300a and 300b can be inserted through each of the holes.

The height restoration instruments 300a and 300b each include a proximal portion 310 and a distal portion 320. The proximal portion 310 comprises a shaft that can serve as a handle to guide a height restoration instrument to a desired location within the vertebral body. The distal portion 320 comprises a flat, pronged or forked end that is wider than the proximal portion 310. In some embodiments, the height restoration instruments 300a and 300b resemble a spatula-shape. In other embodiments, the height restoration instruments 300a and 300b are uniform throughout their length.

Once the height restoration instruments 300a and 300b are placed in a desired position within the vertebral body 1, the instruments can be used to restore the height of the vertebral body 1. While in the illustrated embodiment, two instruments 300a and 300b are used to restore the height, in other embodiments, a single instrument or three or more instruments can be provided through one or more access holes through the vertebral body.

FIG. 6C illustrates the height restoration instruments 300a and 300b restoring the height of the vertebral body 1. The height restoration instruments 300a and 300b can work together to restore the height of the vertebral body. As shown in FIG. 6C, height restoration instrument 300a is angled to press up against the top surface of the vertebral body 1, while height restoration instrument 300b is angled to press down against the bottom surface of the vertebral 1, thereby advantageously distracting and restoring height of the vertebral body 1. In some embodiments, the instruments 300a and 300b work simultaneously. In addition, while not illustrated, a connecting mechanism (e.g., a cuff member) can be provided that connects the two instruments 300a and 300b within the vertebral body.

FIGS. 7A-7E illustrate an alternate method of vertebral body height restoration according to some embodiments. While the illustrated alternate method utilizes height restoration instruments 300a and 300b as in the method described in FIGS. 6A-6C, additional steps using additional instruments are performed during the alternate height restoration process as discussed below.

FIG. 7A illustrates the step of introducing a cavity creation instrument 400a into an access hole 6a of the vertebral body 1. The cavity creation instrument 400a is introduced through a hole 6a formed in the pedicle 2a of the vertebral body 1. While the access hole 6a is formed through the pedicle, access holes can also be formed in other parts of the vertebral body, including extra-pedicularly.

The cavity creation instrument 400a includes a proximal portion 410 and a distal portion 420. The proximal portion 410 comprises a shaft that can form part of a handle to guide or steer the cavity creation instrument 400a to a desired location within the vertebral body 1. The distal portion 420 can comprise an expandable member that can assume an expanded form during rotation that cuts and creates a void within the vertebral body. In some embodiments, the distal portion 420 includes one or more cavity creating elements, including cutting elements (e.g., blades) that help to cut away at bone within the vertebral body 1 to form a cavity or void therein.

FIG. 7B illustrates the step of forming a cavity using the cavity creation instrument 400a. The distal portion 420 of the cavity creation instrument 400a is expanded such that the cavity creating elements are expanded and capable of forming the cavity. Actuating motion of the cavity creation instrument 400a via its shaft causes a cavity to be formed within the vertebral body 1. The actuating motion can include rotation, poking, pushing, scraping back and forth, and other types of movement.

FIG. 7C illustrates the step of forming a cavity using a second cavity creation instrument 400b in addition to the cavity creation instrument 400a. The second cavity creation instrument 400b is introduced through a second hole 6b formed in the second pedicle 2b. Like the first cavity creation instrument 400a, actuating motion of the second cavity creation instrument 400b can assist in forming a cavity within the vertebral body. Once the cavity is formed, height restoration instruments 300a and 300b can be provided to distract and restore the height of the collapsed vertebral body 1.

FIG. 7D illustrates the step of introducing the height restoration instruments 300a and 300b to restore the height of the collapsed vertebral body. As shown in FIG. 7D, the height restoration instruments 300a and 300b are advantageously introduced through the same access holes formed in the pedicles 2a and 2b through which the cavity creation instruments 400a and 400b were introduced.

FIG. 7E illustrates the step of distracting the vertebral body 1 using the height restoration instruments 300a and 300b. As shown in FIG. 7E, height restoration instrument 300a is angled to press up against the top surface of the vertebral body 1, while height restoration instrument 300b is angled to press down against the bottom surface of the vertebral 1, thereby advantageously distracting and restoring height of the vertebral body 1. After performing a distraction to restore height of the vertebral body, the height restoration instruments 300a and 300b can be removed. Following cavity creation and distraction, in some embodiments, a bone filler material can be added to fill the interior of the vertebral body.

FIG. 7F illustrates the step of adding bone filler material 430 into the interior of the vertebral body 1. One of more injection devices can be provided to advantageously inject bone filler material 430 into the vertebral body. The vertebral body with restored height is thus advantageously filled with bone filler material, thereby adding support to the interior of the vertebral body.

While the methods described in FIGS. 6A-7F are with respect to vertebral bodies, one skilled in the art will appreciate that similar methods can also be performed with respect to disc spaces. For example, a pair of height restoration instruments can also be inserted into a disc space through two openings to distract the disc space and restore disc height.

It will be apparent to those skilled in the art that various modifications and variations can be made in the present invention without departing from the scope or spirit of the invention. Moreover, the improved bone screw assemblies and related methods of use need not feature all of the objects, advantages, features and aspects discussed above. Thus, for example, those skilled in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed bone screw assemblies. Thus, it is intended that the present invention cover the modifications and variations of this invention provided that they come within the scope of the appended claims or their equivalents.

What is claimed is:

1. A method for height restoration of a vertebral member comprising:
forming a first access hole through a vertebral member;
inserting one or more modular spacers through the first access hole to restore height of the vertebral member;
forming a second access hole through the vertebral member; and
inserting one or more modular spacers through the second access hole to restore height of the vertebral member.

2. The method of claim 1, wherein the vertebral member comprises a vertebral body.

3. The method of claim 1, wherein the vertebral member comprises a disc.

4. The method of claim 1, wherein at least two modular spacers are placed through the first access hole to restore height of the vertebral member.

5. The method of claim 4, wherein at least two modular spacers are placed through the second access hole to restore height to the vertebral member.

6. The method of claim 4, wherein the at least two modular spacers are stacked against each other within the vertebral body.

7. The method of claim 1, wherein the one or more modular spacers through the first access hole are positioned against the vertebral member on a superior and/or inferior surface.

8. The method of claim 1, wherein the one or more modular spacers through the first access hole are positioned against the vertebral member on an anterior and/or posterior surface.

9. The method of claim 1, wherein the first access hole is formed through a pedicle of the vertebral member.

10. The method of claim 1, wherein the first access hole is formed outside of the pedicle of the vertebral member.

11. A method for height restoration of a vertebral member comprising:
forming a first access hole through a vertebral member;
inserting at least two modular spacers through the first access hole to restore height of the vertebral member and create a structural framework within the vertebral member; and
injecting bone filler material into the vertebral member.

12. The method of claim 11, wherein the first access hole is formed through the pedicle.

13. The method of claim 11, wherein the at least two modular spacers are stacked in contact with each other.

14. The method of claim 11, wherein at least three modular spacers are inserted through the first access hole.

15. The method of claim 11, wherein the at least two modular spacers have a width of between about 1 mm and 11 mm, and a length of between about 5 mm and 90 mm.

16. A method for height restoration of a vertebral member comprising:
forming a first access hole through a vertebral member;
inserting at least two modular spacers through the first access hole to restore height of the vertebral member and create a structural framework within the vertebral member, wherein the at least two modular spacers are in contact with each other within the vertebral member; and
injecting bone filler material into the vertebral member.

17. The method of claim 16, wherein the first access hole is formed through a pedicle of the vertebral member.

18. The method of claim 16, wherein the first access hole is formed through an outer surface of a disc space.

19. The method of claim 16, further comprising inserting a cavity creation instrument through the vertebral member to form a cavity therein before inserting the at least two modular spacers.

20. The method of claim 19, wherein the cavity creation instrument comprises one or more blades.
